(19) United States
(12) Patent Application Publication
(10) Pub. No.: US 2020/0229975 A1
(43) Pub. Date: Jul. 23, 2020

(54) DEVICES AND METHOD FOR PREPARING AND CARRYING OUT CORNEAL TATTOOS

(71) Applicant: NEORIS, Paris (FR)

(72) Inventors: Francesco FERRARI, Strasbourg (FR); Mark BISCHOFF, Jena (DE)

(21) Appl. No.: 16/619,657

(22) PCT Filed: Jun. 7, 2018
(86) PCT No.: PCT/FR18/51336

§ 371 Date: Dec. 5, 2019

(30) Foreign Application Priority Data
Jun. 7, 2017 (DE) 10 2017 209 574.1

(51) Int. Cl. A61F 9/008; A61F 9/00

(52) U.S. Cl. CPC A61F 9/0084; A61F 9/00836; A61F 9/0017; A61F 2009/00897; A61F 2009/00872; A61F 2009/0088; A61F 2009/00878

(57) ABSTRACT

The planning device for determining control data for a treatment device enables surgical tattooing of the cornea of an eye. Based on the measurement data and functional data introduced, a substantially annular surface is defined, which is located inside the cornea and which is delimited by a circular internal edge with an interior diameter and by a circular external edge with an exterior diameter. The annular surface has a distance and an incline with regard to the surface of the cornea, and for this annular surface, a set of control data for controlling the laser device is generated which defines a pattern of target points in the cornea with perforation zones partially or completely intersecting. Upon application of the pulsed laser beam, the tissue of the cornea is cut, and the external edge of the annular surface has a constant distance relative to the external edge of the iris.

### CROSS-REFERENCE TO RELATED APPLICATIONS

See Application Data Sheet.

### STATEMENT REGARDING FEDERALLY SPONSORED RESEARCH OR DEVELOPMENT

Not applicable.

### THE NAMES OF PARTIES TO A JOINT RESEARCH AGREEMENT

Not applicable.

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not applicable.

### STATEMENT REGARDING PRIOR DISCLOSURES BY THE INVENTOR OR A JOINT INVENTOR

Not applicable.

### BACKGROUND OF THE INVENTION

### 1. Field of the Invention

The present invention relates to a planning device for determining control data for a treatment device enabling surgical tattooing of the cornea of an eye of a patient, a corresponding treatment device, and a method for preparing and generating control data for a treatment device for the surgical tattooing of the cornea of an eye of a patient, as well as methods for the surgical tattooing of the cornea of an eye.

### 2. Description of Related Art Including Information Disclosed Under 37 CFR 1.97 and 37 CFR 1.98.

The iris is an anatomical structure in the front part of the eye of a human being. The most important function of the iris is to control the size of the pupil. This process takes place unconsciously due to the brightness of the image produced on the retina. A lesser-known function consists in that the size of the pupil is also unconsciously reduced during the accommodation reflex and contributes, with accommodation (variation of the curvature of the crystalline lens) and the convergence, to the near vision. Furthermore, the human iris has an individual color and structure, and is therefore of great importance from an esthetic perspective.

A functional disruption to the vision process, more particularly a glare phenomenon, generally produces a deviation from the normal working of the iris. A deviation of the visual appearance of the iris from the norm or the ideal of individual beauty may cause suffering.

A change to the iris with surgical methods is among the most demanding objectives in eye surgery. Such procedures most often take place for reconstruction following injuries, then for correction, both functional and cosmetic. Surgery of the iris is linked to a whole series of medical risks and is done relatively rarely, and most of the time only by specialized ophthalmology surgeons, who have extensive knowledge and equipment for acceptable management of complications.

Iris surgery can be avoided in many cases when, in its place, appropriate tattooing is done in the cornea. To that end, during a surgical procedure of the cornea, corresponding pigments are introduced into the cornea. This results, in the cornea, in a pupil that is surrounded by the iris constituted by the pigments. Such a corneal iris resembles the natural iris both from a functional and a cosmetic standpoint. Its cosmetic appearance does not allow it to be distinguished from the natural iris except during precise observation.

“Conventional” methods exist for corneal tattoos, such as the method used and described by Jorge L. Alio under the name Superficial Automated Keratopigmentation (SAK) (J. L. Alio et al., “Femtosecond-assisted keratopigmentation for functional and cosmetic restoration in essential irisatrophy”, Br J Ophthalmol 2010 94: 245-249). The pigment is applied with a micropunctural device (Vissum Eye MP System, micropunctural device) in the cornea of the eye. This method adapts the conventional tattooing technique to the performance of a corneal tattoo and therefore has clearly artisanal and artistic aspects. Granted, it involves a safer and more effective method from a medical standpoint, but the predictability precision regarding the cosmetic result depends greatly on the skill of the surgeon. Such a method is therefore not right for many users or patients.

A similar method, which can also be used on opaque corneas, is described by Jin H. Park et al. (Jin H. Park et al., Int J Ophthalmol. 2015; 8(5): 928-932). A dye in suspension containing a carbon black is used, which is first sterilized in an autoclave. The dye contains carbon black pigments with a size of 200 nm. During observation using a surgical microscope, the dye is injected into the anterior stroma using an injection syringe through a transepithelial route, that is to say, without removing the epithelium. The tattoo is then done conventionally with distances between the pricks of about 1 mm, an injection surface with a diameter of about 1.5 to 2 mm being filled at each prick. After each prick, rinsing is done with a physiological saline solution. The number of pricks varies as a function of conditions that the operator observes in the cornea, but is not greater than 30. Upon each prick, it is necessary to be very cautious so as not to perforate the cornea. After the operation, a therapeutic contact lens must first be worn on the eye.

### BRIEF SUMMARY OF THE INVENTION

The aim of the present invention is therefore to describe the devices and methods for preparing and performing corneal tattoos in order to produce an artificial iris, which avoid the problems mentioned above and which make it possible to create an artificial iris in the cornea that can barely be distinguished from the natural iris and that fulfills, from a medical perspective, a maximum number of functions fulfilled by the natural iris, with few side effects.

The present invention is defined by claim 1 as well as by the secondary claims.

This objective is achieved by the inventor owing to its own practice of a corneal tattooing method, as for example described in document US 2014/0107631: in a first step, with a fs laser system, an annular cut is made inside the cornea and at least one access cut (incision) is made. The tissue bridges still existing are manually separated using a surgical tool (for example a flap lifter), which is introduced for that purpose through the incision into the annular cut. A liquid solution of a dye with an appropriate pigment is next introduced into the annular cut thus prepared. The pigments remain in the tissue and cause permanent coloration, which is only partially reversible with the dyes currently available by later rinsing.

At least one access cut is made with the laser system. However, currently, two access cuts are generally made, which extend in the radial direction over the entire width of the annular cut thereof toward the surface of the cornea.

In this method, which is at the base of the present invention, the ICR option of the VisuMax (treatment apparatus with femtosecond laser for refraction corrections by Carl Zeiss Meditec™, a possibility of making precise cuts inside the cornea of an eye is used to produce an annular cut surface, therefore ultimately an annular tunnel inside the cornea. This annular cutting surface is characterized by an interior diameter and an exterior diameter as well as a distance, characterizing its depth in the cornea, and its incline relative to the surface of the cornea. The lateral positioning takes place, taking account of optical considerations, in a centered manner relative to an identified point on the surface of the cornea, for example at the passage point of the visual axis or the axis of the pupil.

If one wishes to make an artificial iris in the cornea of an eye of a patient, for cosmetic reasons, in order to obtain a desired color of the iris or for medical reasons to replace the working of a damaged iris as completely as possible, and to approach a natural iris as closely as possible, a series of conditions should be met:

The human eye does not have a perfectly symmetrical structure; for example, there is no optical axis technically speaking. The various optical elements of the eye are still considerably loosened relative to one another, they are not perfectly round, that is to say, there is no exact rotational symmetry. To date, the interior edge as well as the exterior edge of the annular cutting surface, in which the tattoo must be made, are still made perfectly circular. This does not have a natural effect when one looks at the eye precisely.

The iris is not only characterized by a particular color, but also by a particular structure. The conventional methods make it possible to obtain only any homogeneous coloring, but they do not make it possible to imitate the fine structure of the coloring of a natural iris during corneal tattooing.

Corneal tattooing is not movable, contrary to the natural iris. Certain ophthalmological exams or treatments (for example, an iridotomy), however, require the mobility of the natural iris to open it in order to obtain better access to the posterior part of the eye. Most of the time, this takes place using appropriate pharmaceutical products (for example, atropine). This is not possible in the case of a corneal tattoo, which is more particularly a problem in the case of an iridotomy, since the method cannot be executed appropriately later.

The access cuts currently extend in the radial direction over the entire width of the annular cut. They are therefore located in the presurgical optical zone and depart radially outward from the pupil made by the corneal tattoo. They therefore touch the same reduced postoperative optical zone during the operation.

If the optical action of an artificial pupil is desired, such a corneal pupil is currently made with a corneal inlay (for example KAMRZ™ by AccuFocus™. This is done in order to improve the near vision, the magnification of the depth of field being done using a very small invariable pupil. The implants used are very difficult to manufacture, and their implantation often considerably affects the metabolic processes in the cornea. It may sometimes result in significant health risks. As a result, explanations are often necessary after some time.

The described objective is achieved, according to the present invention, using a planning device to determine control data for a treatment device for the preparation of a surgical tattoo of the cornea of an eye of a patient. This planning device is in particular based on data derived from measurements of parameters of the eye to be operated and functional data reflecting cosmetic or medical functions applicable to the eye to be operated. All of this data is taken into account, in the invention, in order to define a zone to be operated, and to control the laser device performing the operation.

The application of a laser beam on a target zone to be operated is known-it is for example disclosed in document US 2015/0305927-but not on a target zone obeying the same structural and geometric characteristics as those of the invention, and also not based on parameters as mentioned above.

In short, the planning device of the invention is designed to generate the control data as defined, that is to say, taking account of a medical and personal reality linked to a patient, intended for a treatment device, said treatment device comprising a laser device that cuts the tissue of the cornea by emitting a pulsed laser beam.

Such a treatment device to that end preferably contains a laser beam source for generating a pulsed laser beam, for example a femtosecond or picosecond laser beam, a lens for focusing laser pulses in the cornea, an x-y scanning system as well as a z scanning system and a control system, which controls the laser beam source, the lens and the scanning systems. The laser beam is focused on target points found in a pattern on the cornea.

According to the present invention, the planning device comprises an interface for introducing measuring data regarding parameters of the eye and functional data for functions to be fulfilled by the tattoo of the cornea of the eye. The functional data indicate which cosmetic and/or medical functions or parameters must be obtained with the production of an artificial iris using the surgical tattoo of the cornea; they therefore also contain geometric requirements or geometric parameters that are extracted from the indicated functional data.

The planning device defines, from the introduced measuring data and functional data, a globally annular surface that is located inside the cornea and that is limited by a globally circular interior edge with an interior diameter and a globally circular exterior edge with an exterior diameter, the interior diameter and the exterior diameter being positioned at an identified position and/or an identified structure on the surface of the cornea, and the annular surface having a distance and an incline relative to the surface of the cornea. The globally annular surface can be flat or curved. This annular surface must constitute, after the end of the method, the tattooed surface, that is to say, it must contain a pigmented dye.

The planning device produces, for this globally annular surface, a set of control data for the control of the laser device, which determines a pattern of target points in the cornea, which are located in the annular surface and which are arranged such that the annular surface is made, after application of the pulsed laser beam according to the set of control points, in the form of a cutting surface.

This annular surface contains, at each target point, a perforation zone in which, during the application of the pulsed laser beam, the tissue of the cornea is separated. This perforation zone is therefore a zone in which the tissue is separated owing to the photo-disruption action of the pulsed laser in the focusing zone of its laser beam. This zone is also called focused action zone.

Perforation zones of adjacent target points can overlap, or cross, partially or completely, a partial overlap or crossing of the perforation zones meaning that, between these zones, tissue bridges remain, while a complete overlap or crossing does not leave any tissue bridge, such that it results in a cutting surface completely passing through a large surface area.

The exterior edge of the globally annular surface has a constant distance from a macroscopic perspective relative to the exterior edge of the iris. The exterior edge is therefore centered relative to the corneal margin. The distance relative to the exterior edge of the iris can also be equal to zero: for the cosmetic aspect, it is important to keep the distance between the exterior edge of the final tattoo and the exterior edge of the iris approximately constant. This is why the cut is preferably centered relative to the corneal margin, and the distance between the exterior edge of the tattoo and the exterior edge of the iris is kept constant in the macroscopic sense, the shape of the exterior edge therefore being able to be different from the ideal round shape, therefore slightly elliptical or oval. Keeping the distance constant in the macroscopic sense means that the exterior edge can contain a fine microscopic structure, the distance of which relative to the exterior edge of the iris varies significantly. However, if a smoothing function is superimposed thereon, it results in a structure of the exterior edge with a constant distance relative to the (macroscopic) exterior edge of the iris.

This measurement causes a visual accentuation in the eyes of the observer and increases the esthetic effect.

The (globally) annular surface determined by the planning device is further designed, advantageously, to absorb a pigmented dye after application of the pulsed laser beam. The usable pigmented dyes (tattoo, tattoo dye) are for example: BioChromaEyes (TM registered) by the manufacturer Laboratoires BIOTIC Phocea (Marseille, France). They are available in many shades. In order to guarantee an optimal tattoo of the annular surface, the production of the annular surface and therefore of all of the control data for the control of the laser device, which determines a pattern of the target points in the cornea, which are located in the annular surface and which are arranged such that the annular surface is made, after the application of the pulsed laser beam according to the data set, in the form of a cutting surface, are adapted to the properties of the pigmented dye, more particularly to its distribution properties in the produced annular cutting surface.

In one design, the perforation zones made during the application of the pulsed laser beam can absorb pigmented dyes, preferably colored microparticles in liquid solution, without additional treatment of the annular cutting surface. In another design, in the annular surface, a complete separation of the tissue zones above and below the annular surface is first done by mechanical separation of the tissue bridges still existing between the perforation zones. In a third design, the perforation zones intersect so strongly that no tissue bridge remains between these tissue zones. If a manual perforation is necessary, this can be done using a surgical tool such as a flap lifter, which is for example introduced using an access cut.

The (globally) annular surface determined by the planning device comprises, in one preferred embodiment, after the application of the pulsed laser beam, a different perforation in different zones of the annular surface. This different perforation is preferably determined as a function of the desired degree of coloring of the tattoo in different zones of the annular surface.

It has been demonstrated that the coloring of the tissue takes place as a function of the thickness of the perforation. A less perforated cutting zone, which requires, during the manual operation, after the laser treatment, a more intensive treatment with the spatula during the finishing of the laser cutting, in order to completely separate the layers of tissue from one another, has a different shade after the treatment and healing. The corneal cuts are therefore made using the laser device of this embodiment of the invention, such that the perforation of the cuts takes place differently in the different zones. This makes it possible to obtain a fine structure of the corneal tattoo, which appears to be particularly natural in the eyes of an observer. More particularly, a radial pattern structure must thus be produced.

The (globally) annular surface determined by the planning device is, in another embodiment, designed and the pigmented dye is produced such that, after the application of the pulsed laser beam and after the absorption of the pigmented dye, in the annular surface, the latter has an absorption greater than or equal to 50%, preferably an absorption greater than or equal to 80% of the visible light. The annular cutting surface, for example regarding its pattern of target points of the focused laser beam, but also regarding the parameters of the laser itself, is adapted to the pigmented dye used, such that a desired absorption effect is obtained in the domain of the visible spectrum.

In another preferred embodiment, the annular surface determined by the planning device comprises a spared zone that is preferably located on the exterior edge of the annular surface; the spared zone further preferably having a surface area greater than 0.2 mm² and preferably less than 5 mm² and ideally comprising an arc-shaped edge. In order to preventively guarantee the accessibility of the natural iris for the laser beam of a therapeutic laser, a zone necessary to that end is spared, and therefore a spared zone is produced. This zone is preferably located at the top of the globally annular surface. An iridotomy can thus be done later, appropriately.

In one design of the planning device, in which a corneal tattoo must be produced for cosmetic reasons and without effect on the vision, the annular surface produced by the latter using corresponding control data comprises an interior edge with an interior diameter greater than 4 mm: in order to avoid visual problems, for example due to an off-centered pupil, the size of the interior edge of the corneal cut is chosen such that the iris formed by the tattoo, and therefore the corneal pupil, has no effect on the vision. This compromise, however, proves problematic when a determined visual effect must be obtained.

In one alternative design of the planning device, in which a visual effect must be obtained and no artificial pupil must be produced, the annular surface generated by the latter using corresponding control data has an interior edge with an interior diameter smaller than 3 mm.

During the use of an appropriate dye, which causes, during use, an absorption of preferably at least 50% of the visible radiation, an optical effect is thus obtained in the form of an additional artificial pupil: this corneal pupil allows improved vision by increasing the depth of field. The effect resembles that of a corneal inlay for the improvement of the near vision. However, it does not involve a fixed implant, but only a tattoo made up of inappropriate pigmented dye (therefore colored microparticles, which are found in a liquid solution), which have a smaller impact on the metabolism inside the cornea of the eye than a solid body. In this embodiment of the invention, the annular cutting surface is produced by the laser device such that the interior edge of this annular cutting surface, and therefore, after the introduction of the pigmented dye, the interior edge of the corneal tattoo has a globally round shape. The surface area of the pupil represents, in order to obtain a corresponding effect on the vision, less than 7 mm². Preferably, the pupil has a surface area of about 2 mm². The absorption of the visible radiation through the corneal tattoo after the introduction of the pigmented dye preferably represents at least 50%, more particularly preferably at least 80%.

In another embodiment, the annular surface determined by the planning device is characterized in that the center of the circular interior edge and the center of the circular exterior edge do not coincide, and therefore the center of the edge is therefore in a (lateral) position different from that of the center of the exterior edge. The center of the interior edge is therefore positioned in order to obtain an optimized vision for the patient, preferably centered on an identified point on the surface of the cornea, for example at the passage point of the visual axis or the axis of the pupil. The center of the exterior edge is positioned in order to obtain a cosmetic result for the patient, for example with a uniform distance relative to the edge of the natural iris.

In one design, the annular surface determined by the planning device is characterized in that the exterior edge and/or the interior edge does not have any smooth curve. Instead of this, the exterior edge and/or the interior edge preferably has apparently random modulations. One thus obtains a particularly natural appearance of the exterior edge and/or the interior edge in the eyes of an observer.

In one embodiment that is designed differently, the planning device is characterized in that it defines, from introduced measurement and functional data, another annular surface that is located inside the cornea, the henceforth at least two annular surfaces having a different distance relative to the surface of the cornea, and these at least two annular surfaces are superimposed, the annular surface that has the smallest distance relative to the surface of the cornea preferably having the smallest gap between the exterior diameter and the interior diameter and the at least two annular surfaces are centered relative to one another regarding the interior edges or regarding the exterior edges.

In this special design of the invention, the laser device therefore produces at least two annular cutting surfaces in the cornea of an eye, such that they are superimposed. The incident light must then penetrate more than one tattoo layer in order to reach the retina. The layers can have different shapes and be positioned differently. The same pigmented dyes or dyes with different pigments can be used for the tattoo. The tattoos therefore fulfill a common optical function, for example a stepped optical absorption or variable as a function of the spectrum. In one special embodiment, a tattoo with a circular interior edge of 1.5 mm in diameter and an absorption of 70% can for example be completed by a second tattoo with a circular interior edge with a diameter of 2 mm and an absorption of 70%. The first tattoo is for example located at a depth of 180 μm and the second tattoo at a depth of 140 μm below the surface of the cornea. In the superposition, this results in a stepped pupil with 100% transmission in the interior zone (diameter of 1.5 mm), 70% transmission between 1.5 mm and 2 mm and 9% transmission beyond 2 mm.

One particularly preferred design of the planning device further defines, from introduced measuring and functional data, at least one access surface, which goes from the surface of the cornea toward the annular surface, and generates, for this access surface, a set of control data for controlling the laser device, which traces, in the cornea, a pattern of target points, which are found in the access surface and which are arranged such that the access surface is made, after the application of the pulsed laser beam, according to the set of control data, in the form of an access cutting surface, the target points of the access surface having a radial distance relative to the center of the interior edge that is greater than half of the interior diameter of the annular surface.

The access cuts must therefore be made such that their interior limitation does not reach the interior edge of the corneal tattoo, so as not to disrupt the operation as such of the pupil of the iris made artificially in the cornea of an eye. Two access cuts per annular surface are typically made; in one variant of the invention, more than two access cutting surfaces are perforated.

In another design of the planning device, the access surface is oriented radially and its radial extension is kept smaller than the gap between the half of the exterior diameter and the half of the interior diameter or the access surface is oriented along the exterior edge or parallel to the exterior edge. To that end, it is cut in an arc shape more particularly along the exterior edge or parallel to the exterior edge.

In one design, the annular surface determined by the planning device is characterized in that it is made after the application of the pulsed laser beam for the housing of an implant.

Another important variant of the planning device is characterized in that the annular surface is defined in a lenticule in the tissue of the cornea of the eye, which is explanted after the application of the pulsed laser beam.

In this variant of the invention, a corresponding annular cutting surface is prepared in a lenticule of a corneal tissue to be transplanted of an eye, such that it is located inside the lenticule, therefore inside the volume of tissue to be explanted. This annular cutting surface located inside the volume of tissue preferably comprises, in a determined location, a particular characteristic, for example a recess. If the position is chosen such that a thickness modulation of the transplant that is difficult for the operator to recognize is identified, therefore for example the axis of the astigmatism in the case of a lenticule removed during a SMILE operation, it is possible to obtain an improvement of the precision during the orientation of the transplant in the recipient eye. To that end, hereinafter, for example after the explantation, a pigmented dye is introduced into the cut, which can serve an esthetic and/or functional medical purpose. In such an embodiment variant of the invention, a lenticule, which has been removed from a patient in order to correct his myopia with an astigmatism, is equipped with an interior annular cutting surface that for example has an interior diameter of about 2 mm and an exterior diameter of 6 mm and has, on the exterior circumference, a recess where the optical axis of the astigmatism is found. During the implantation in an appropriate hypermetropic eye with an astigmatism, the lenticule is oriented such that the recess is oriented appropriately relative to the axis of the astigmatism to be corrected. The precision of the correction of the astigmatism is thus improved and the pupil function of the tattoo further increases the depth of field of the eye simply. With another marking on the lenticule, it is further possible to obtain a lateral recognition of the lenticule during the transplant. This can be important more particularly when the lenticule is treated between the explantation and the implantation, for example in order to clear it of cells (for example by radiation) or to make a change to the shape (for example, removal of the surface).

In one advantageous design, the planning device is characterized in that the annular surface is positioned using a recorded image. In order to check the correct positioning of the annular cutting surfaces or other cutting surfaces to be made with the laser device and to avoid any shift, it is advantageous to position the annular surface or the other cutting surfaces to be made using a recorded image. To that end, it is possible to use a recording using the corneal margin or using the pupil.

Furthermore, it is advantageous for the planning device to be connected to the interface of a measuring device that generates the measuring data coming from a measurement of the eye and introduces them into the planning device, the measuring device optionally comprising one or several of the following devices: autorefractometer, refractometer, keratometer, aberrometer, wave front measuring device, optical coherence tomograph (OCT).

It is also advantageous for the planning device for a datalink or data medium to be provided for transmitting the set of control data via the planning device to the laser device or the entire treatment device. This makes it possible to guarantee the systematic transmission of the data that must ultimately make it possible to control the application of the pulsed laser beam to the cornea of the eye by means of the treatment device.

A display device for the visual representation of control data and an input device for the subsequent modification of the set of control data that are provided in one advantageous design of the planning device, facilitate the determination of the control data for a treatment device allowing the surgical tattoo of the cornea of an eye of a patient.

One preferred planning device is further characterized in that the planning device takes account, during the generation of the set of control data that contains the pattern of the target points, of the deformation of the cornea of the eye, which occurs during the application of the pulsed laser, more particularly using an interface with the patient, optionally a contact lens or a liquid interface with the patient, using which the position of the eye of a patient is typically fixed relative to a treatment device for the surgical tattoo. After the detachment of the fixing, the annular surface is then in the nondeformed cornea.

The aim of the invention is further achieved using a treatment device for a surgical tattoo of the cornea of an eye of a patient, which comprises an interface for introducing measuring data regarding the parameters of the eye and functional data regarding the functions to be fulfilled by the tattoo of the cornea of the eye, a laser device that cuts the tissue of the cornea by applying a pulsed laser beam, the laser beam being focused on target points defining a pattern in the cornea, and a planning device described hereinabove.

More particularly, such a treatment device to that end preferably contains a laser device that comprises a laser beam source for producing a pulsed laser beam, for example a femtosecond laser beam or a picosecond laser beam, a lens for focusing laser pulses on the cornea, an x-y scanning system and a z scanning system and a control system that controls the laser beam source, the lens and the scanning systems.

The treatment device is preferably a femtosecond laser keratoma for making cuts inside the cornea of an eye, with a lens for focusing laser pulses on the cornea and an x-y scanning system and a z scanning system. The treatment device is further provided with a system for controlling the focal length, preferably the energy of the laser pulses being able to be controlled, more particularly the pulses being able to be activated and deactivated on the path of the scanning. It is thus possible, for example, to give the spared zone nearly any shape and size. The modulation of the proximity of the exterior edge of the corneal annular cutting surface for example also makes it possible to modulate the exterior edge.

The aim of the invention is further achieved using a method for preparing and generating control data for a treatment device allowing the surgical tattoo of the cornea of an eye of a patient, the treatment device comprising a laser device that cuts the tissue of the cornea by applying a pulsed laser beam and the laser beam focusing, during its operation, the laser beam according to the control data on target points located in a pattern in the cornea. This method is characterized by the following steps:

determining measuring data regarding the parameters of the eye and functional data regarding the function to be fulfilled by the tattoo of the eye,

defining a globally annular surface, flat or, if applicable, also curved, from measuring data and functional data,
the annular surface being located inside the cornea and being limited by a globally circular interior edge with an interior diameter and a globally circular exterior edge with an exterior diameter,
the interior diameter and the exterior diameter being positioned relative to an identified point and/or an identified structure on the surface of the cornea, and
the annular surface having a distance and an incline relative to the surface of the cornea,

defining a pattern of target points in the cornea,
the target points being located in the globally annular surface and being arranged such that the annular surface is made in the form of a cutting surface according to the control data after the application of the pulsed laser beam,
the annular surface comprising, at each target point, a perforation zone in which, during the application of the pulsed laser beam, the tissue of the cornea is cut, the perforation zones being able to partially or completely cut adjacent target points, and
the exterior edge of the globally annular surface having a constant distance from a macroscopic perspective relative to the exterior edge of the iris,

generating a set of control data containing the two- or three-dimensional pattern for the control of the laser device.

The main properties of the other design of the method are already described in detail in the description of the planning device. They will therefore only be summarized here.

In specific designs, the method is therefore characterized in that the annular surface is further defined or the pattern of target points on the cornea is defined such that at least one of the following statements is relevant:

the target points of the annular surface are arranged such that the annular surface is further made, after the application of the pulsed laser beam, for housing an appropriate pigmented dye;

the target points of the annular surface are arranged such that the annular surface has, after the application of the pulsed laser beam, a different perforation in different zones of the annular surface;

the annular surface comprises a spared zone that is preferably arranged on the exterior edge of the annular surface; the spared zone preferably having a surface area greater than 0.2 mm² and preferably less than 5 mm²;

the annular surface comprises an interior edge with an interior diameter greater than 4 mm or the annular surface comprises an interior edge with an interior diameter smaller than 3 mm;

the center of the circular interior edge and the center of the circular exterior edge do not coincide;

the exterior edge and/or the interior edge do not have a smooth curve;

the annular surface is made, after the application of the pulsed laser beam, for housing an implant;

the annular surface is defined in a lenticule in the tissue of the cornea of the eye, which is explanted after the application of the pulsed laser beam;

the annular surface is positioned using a recorded image.

Furthermore, one preferred design of the method is characterized in that, from measurement data and functional data, at least one other annular surface is defined, which is located inside the cornea, the at least two annular surfaces having different distances relative to the surface of the cornea, and the at least two annular surfaces being superimposed, preferably the annular surface that has the smallest gap between the exterior diameter and the interior diameter having the smallest gap between the exterior diameter and the interior diameter and preferably the at least two annular surfaces being centered relative to one another regarding the interior edges or the exterior edges.

Another preferred design of the method is characterized in that, from measurement data and functional data, at least one access surface is further defined, which goes from the surface of the cornea toward the annular surface, and for this access surface, control data for controlling the laser device are generated, which define a pattern of target points in the cornea, which are found in the access surface and which are arranged such that the access surface is made in the form of an access cutting surface after the application of the pulsed laser beam according to the set of control data, the target points of the access surface having a radial distance relative to the center of the interior edge that is greater than half of the interior diameter of the annular surface.

Optionally, the method is also characterized in that the access surface is oriented radially and its radial extension is smaller than the gap between half of the exterior diameter and half of the interior diameter, or the access surface is oriented along the exterior edge or parallel to the exterior edge.

Advantageously, a method for preparing and generating control data for a treatment device allowing the surgical tattoo of the cornea of an eye of a patient is further characterized in that:

measuring data coming from a measurement of the eye are generated, one or several of the following devices optionally being used as measuring device:
autorefractometer, refractometer, keratometer, aberrometer, wave front measuring device, optical coherence tomograph (OCT), and/or

the generated control data are transmitted to the treatment device; these data being transmitted by means of a data link or a data medium to the laser device.

Since the position of the eye of a patient relative to a treatment device allowing a surgical tattoo is typically fixed, an interface with the patient, optionally a contact lens or a liquid interface with the patient, being used to that end, which, during the securing of the eye to the treatment device, deforms the cornea of the eye, one preferred method for preparing and generating control data for a treatment device allowing the surgical tattooing of the cornea of an eye of a patient is characterized in that, during the generation of the control data, which contain the pattern of target points, a deformation of the cornea of the eye, which occurs during the application of the pulsed laser beam, is taken into account, such that the defined annular surface is found in the cornea subsequently not deformed.

The aim of the invention is further also achieved owing to a software product with a program code, which, during the execution on a computer, executes the method described above for the preparation and generation of control data for a treatment device allowing the surgical tattooing of the cornea of an eye of a patient, as well as owing to a data medium with such a software product.

The aim of the invention is achieved owing to a method for surgical tattooing of the cornea of an eye of a patient, with the following steps:

executing the method for preparing and generating control data for a processing device allowing the surgical tattooing of the cornea of an eye of a patient, which comprises a laser device, which cuts the tissue of the cornea by applying a pulsed laser beam, according to the aforementioned method for preparing and generating control data for a treatment device allowing the surgical tattooing of the cornea of an eye of a patient,

surgical laser treatment of the cornea of the eye with the treatment device using the generated control data,

optional mechanical cutting of the tissue bridges remaining after the laser treatment, in the surfaces made during the surgical laser treatment, for example with a surgical tool such as a flap lifter;

injection of at least one pigmented dye into the annular surface, for example using access surfaces.

### BRIEF DESCRIPTION OF THE SEVERAL VIEWS OF THE DRAWINGS

The present invention is explained using exemplary embodiments.

FIG. 1 is a schematic view of the treatment device with a planning device.

FIG. 2 is a schematic view of elements of the treatment device, more specifically the laser device.

FIG. 3 is a schematic view of a first exemplary embodiment of a corneal tattoo with a spared zone seen from above.

FIGS. 4a and 4b are schematic views of a second exemplary embodiment of a corneal tattoo with different centering of the edges seen from above and in sectional view.

FIGS. 5a and 5b are schematic views of a third exemplary embodiment of a corneal tattoo with two annular surfaces superimposed in top view and sectional view.

FIGS. 6a and 6b are schematic views of a fourth exemplary embodiment of a corneal tattoo in a lenticule in side view and in top view, respectively.

FIG. 7a is a schematic view of penetration zones partially intersecting in a cutting surface.

FIG. 7b is a schematic view of penetration zones fully intersecting in a cutting surface.

### DETAILED DESCRIPTION OF THE INVENTION

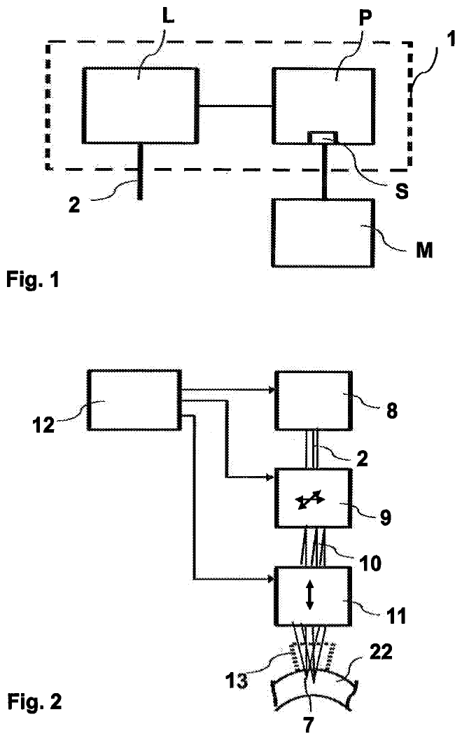

FIG. 1 schematically shows the treatment device 1. In this variant, it comprises at least two devices or modules. A laser device L emits the laser beam 2 toward the eye 3. The operation of the laser device L takes place completely automatically, that is to say, the laser device L begins, upon a corresponding start signal, to point the laser beam 2 and makes cutting surfaces in the cornea 22, which is structured in a manner that remains to be described. The control data necessary for the operation is received beforehand by the laser device L coming from a planning device P in the form of a set of control data by means of data lines not described in detail. The transmission preferably takes place before the operation of the laser device L. Of course, the communication could also take place wirelessly. In a variant with direct communication, it is also possible for the planning unit to be physically separated from the laser unit L and to provide a corresponding data transmission channel.

Preferably, the set of control data is transmitted to the treatment device 1 and preferably an operation of the laser device L is blocked until a valid set of control data reaches the laser device L. A valid set of control data can be a set of control data that in principle is suitable for use with the laser device L of the treatment device 1. However, the validity can be associated with the fact that several checks are successful, for example if, in the control data set, additional information is found regarding the treatment device 1, for example a device serial number, or the patient, for example a patient identification number, that matches other information, which is for example read on the treatment device or has been entered separately once the patient is in a correct position for the operation of the laser device L.

The planning unit generates all of the control data, which is made available to the laser unit L to perform the operation, from measurement data that have been determined for the eye to be treated, as well as functional data for functions to be performed by the tattoo of the cornea of the eye, for example a function to replace the natural iris and the performance of a pupil function. They are introduced into the planning unit using the interface S. In the illustrated example, the measurement data come from a measuring device M, which has previously measured the eye of the patient 4. Of course, the measurement device M can send the corresponding measurement data to the planning unit in any manner whatsoever.

The transmission can take place using memory chips (for example by USB key or memory stick), magnetic memories (for example, discs), by radio (for example, WLAN, UMTS, Bluetooth) or by cable (for example USB, FireWire, RS232, ADC bus, Ethernet, etc.). The same is of course valid regarding the transmission of data between the planning device P and the laser device L.

A direct radio or cable link of the measuring device M with the treatment device 1 regarding the data transmission, which can be used in a variant, has the advantage that the use of incorrect measuring data is precluded with the highest possible likelihood.

FIG. 2 introduces elements of the treatment device 1 only to the extent that they are necessary to understand the adjustment of the focus. The laser beam 2 is focused in a focal spot 7 in the cornea 22, and the position of the focal spot 7 in the cornea 22 is adjusted such that, to produce the cutting surface, energy coming from pulses of laser beams is applied in a focused manner in different locations of the tissue of the cornea 22. The laser beam 2 is provided by a laser 8 in the form of a pulsed beam. The cornea 22 of the eye 3 is fixed using an interface with the patient 13, here more particularly with a contact lens, to the treatment device 1. An x-y scanner 9, which is made, in a variant, using two galvanometric mirrors with globally orthogonal deviations, points the laser beam coming from the laser 8 in a two-dimensional manner, such that a laser beam 10 exists after the x-y scanner 9. The x-y scanner 9 therefore allows an adjustment of the focal spot 7 in a manner globally perpendicular to the main incidence direction of the laser beam 2 in the cornea 22. For the adjustment of the depth position, in addition to the x-y scanner 9, a z scanner 11 is provided, which is for example designed as an adjustable telescope. The z scanner 11 makes it possible to modify the z position of the focal spot 7, that is to say, its position on the optical axis of the incidence. The z scanner 11 can be arranged after or before the x-y scanner 9. The coordinates, called x, y, z hereinafter, refer to the deviation of the position of the focal spot 7.

For the operating principle of the treatment device 1, the assignment of the different coordinates to the spatial directions is not important, but in order to facilitate the description, hereinafter, z always designates the coordinate along the optical axis of the incidence of the laser beam 2, and x and y designate two coordinates orthogonal to one another in a plane perpendicular to the direction of incidence of the laser beam. One skilled in the art naturally knows that a three-dimensional description of the position of the focal spot 7 in the cornea 22 can also take place using other coordinate systems; more particularly, it may not involve a system of perpendicular coordinates. It is therefore not mandatory for the x-y scanner 9 to operate around axes that are perpendicular to one another, but any scanner that is able to move the focal spot 7 in a plane in which the incidence axis of the optical radiation is not located can be used. Systems of coordinates at oblique angles or on Cartesian systems of coordinates can therefore also be used.

In order to control the position of the focal spot 7, the x-y scanner 9 as well as the z scanner 11, which together define a concrete example of a three-dimensional focal spot adjusting device, are controlled by a control device 12 by means of lines, which are not described in detail. The same is valid for the laser 8. The control device 12 guarantees an appropriate synchronous operation of the laser 8 as well as the adjusting device of the focal spot, defined for example by the x-y scanner 9 as well as the z scanner 11, such that the position of the focal spot 7 in the cornea 22 is adjusted so that ultimately the annular cutting surface, which can also be curved, and which must later observe the pigmented dyes, is reached by the scanning of predetermined target points and by the application of the pulsed laser beam at these target points.

The control device 12 operates according to predetermined control data, which determine target points for the adjustment of the focal spot. The control data are generally grouped together in a set of control data. The latter determines, in one embodiment, the coordinates of the target points as pattern, the order of the target points in the set of control data defining the succession of the positions of the focal spot, and therefore ultimately a route. In one embodiment, the set of control data contains the target points in the form of concrete adjusting values for the mechanism for adjusting the position of the focal spot, for example for the x-y scanner 9 and the z scanner 11. In order to prepare the ocular surgery method, therefore before the surgical procedure strictly speaking can be carried out, the target points and preferably also their order in the pattern are defined. Prior planning of the procedure must, however, take place by determining, for the treatment device 1, the control data whose use will make it possible to obtain an optimal tattoo of the cornea for the patient 4.

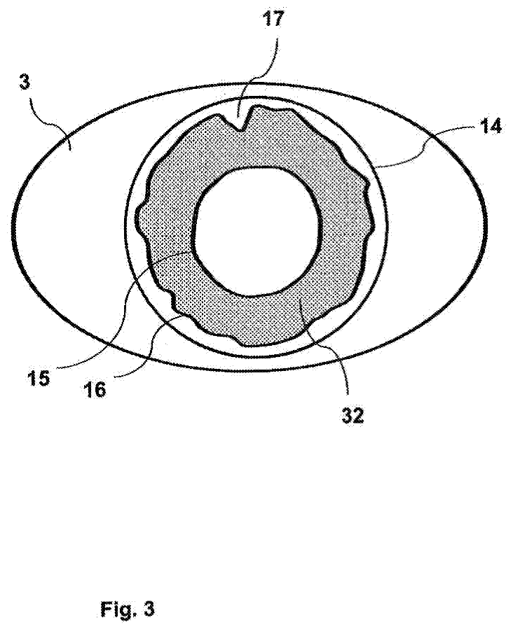

FIG. 3 shows a first exemplary embodiment of a corneal tattoo that contains a spared zone 17, which can be used for an iridotomy with the YAG laser, in top view on the eye 3. In this example, the interior edge 15 has a large diameter of 5 mm and comprises a slightly irregular ridge, since the corresponding corneal tattoo 15 is used solely for cosmetic purposes. The exterior edge 16 comprises a highly irregular ridge in order to represent a natural iris with great precision in the details. After the smoothing of these irregular ridges, in the macroscopic direction, the distance between the exterior edge 16 and the exterior edge of the iris is therefore relative to the lim bus 14 is constant. The spared zone has been made in the upper part of the eye 3, which is generally covered by the eyelid, such that the spared zone 17 is difficult for an observer to see.

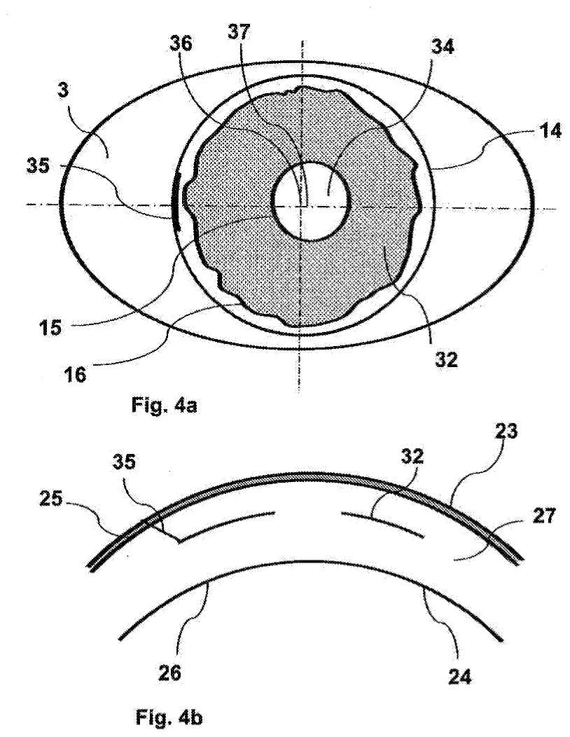

FIGS. 4a and 4b show a second exemplary embodiment of a corneal tattoo on a globally annular surface 32 with a different centering of the exterior edge 16 and the interior edge 15 seen in top view and sectional view. The centering 36 of the exterior edge 16 takes place relative to the limbus 14, while the interior edge 15 has been centered relative to the center 37 of the natural photopic pupil 34. In the sectional view, one can further see the position of the surface of the cornea 23, from behind the cornea 24, the epithelium 25, the endothelium 26 and the stroma 27. An access surface 35 extends from the surface of the cornea 23 toward the surface 32 in which a pigmented dye must be injected. This access surface 35 is arranged parallel and in an arc of circle relative to the exterior edge 16 of the annular surface 32.

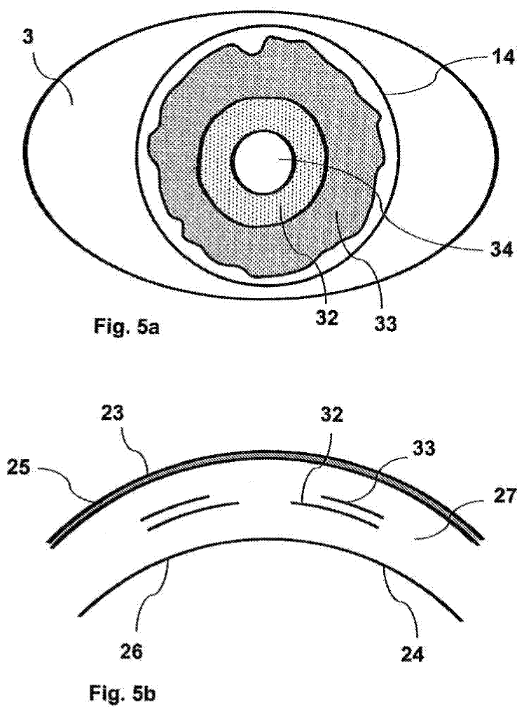

FIGS. 5a and 5b show a third exemplary embodiment of a corneal tattoo with two superimposed annular surfaces 32, 33, that is to say, located one on top of the other, arranged at different distances relative to the surface of the cornea 23, seen in top view and in sectional view. The corneal tattoo is therefore made up of a first tattoo on a first annular surface 32 and a second tattoo on a second annular surface 33, the two tattoos being centered relative to one another regarding their exterior edges 16. The second tattoo is closer to the surface of the cornea 23, that is to say, above the first tattoo, seen from the inside of the eye toward the outside, since it is the narrowest, which has, for the same diameter of the exterior edge 16, a diameter of the interior edge 15 that is much larger than that of the first tattoo. Thus, independent access surfaces 35 (not shown here) both from the first annular surface 32 and the second annular surface 33 can also be made, since the two surfaces 32, 33 remain at least partially visible in top view.

In the exemplary embodiment shown here, the incident light can therefore pass through more than one layer of tattoo to reach the retina. Identical pigments or different pigments can be used for the tattoo. Both tattoos make it possible to obtain a common optical function, for example a stepped or variable optical absorption depending on the spectrum. The tattoo shown here with a circular interior edge 15 of a first tattoo with a very small diameter of 1.5 mm has an absorption of 70%, the second tattoo with a circular interior edge 15 of 2 mm also has an absorption of 70%. The first tattoo is at a depth of 180 μm and the second tattoo is at a depth of 140 μm below the surface of the cornea 23. The superposition makes it possible to obtain a stepped pupil 34 with a transmission of 100% in the inner part (diameter 1.5 mm), a transmission of 70% between 1.5 mm and 2 mm and a transmission of 9% beyond 2 mm.

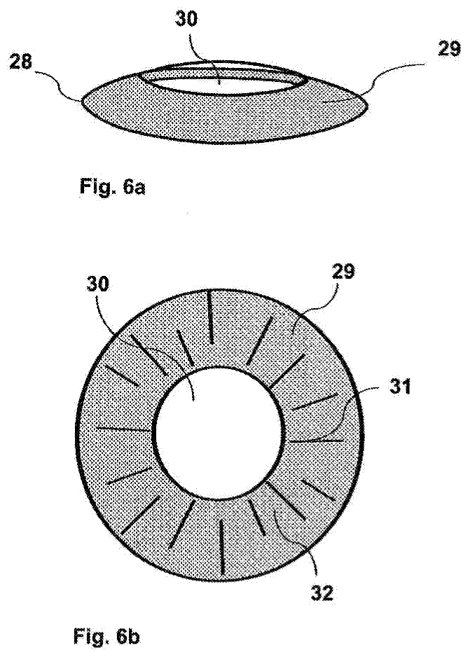

FIGS. 6a and 6b show a fourth exemplary embodiment of a corneal tattoo, which has been done in a lenticule 28 to be explanted, in side view and in top view. This lenticule 28 can then be implanted in the eye 3 of a patient, in which it will simultaneously correct a corresponding vision defect. The lenticule 28 contains a colorless center 30 and is colored 29 annularly in the exterior part, in return for which it has a fine colored structure 31 that has been obtained using a different perforation 6 in different zones of the annular surface 32.

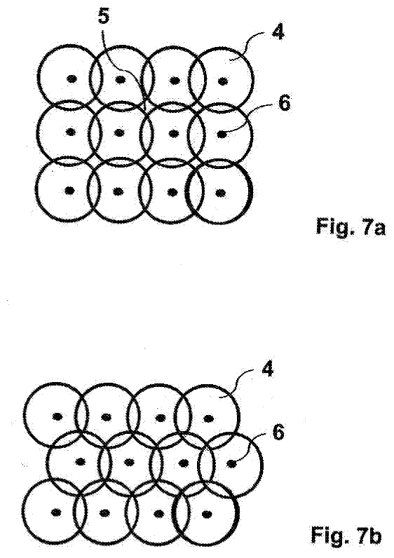

FIG. 7a shows what must be understood as zones with perforations partially crossing one another in a cutting surface: around target points 6 of the focal spot 7 of the pulsed laser beam, it results, during the application of this laser beam 2, in a perforation zone 4, the extension of which depends inter alia on the frequency of the pulsation and the power of the pulsed laser beam. The edges of the perforation zone cross one another here only partially, and tissue bridges 5 remain between the perforation zones 4.

FIG. 7b shows perforation zones 4 crossing one another completely in a cutting surface, such that no tissue bridge 5 remains any longer, and after the application of the pulsed laser beam 2, a through cutting surface, with a variable zone separated by photo-disruption, the tissue of the cornea being located between, above and below the perforation zone 4.

The features of the invention mentioned above and explained in various exemplary embodiments can be used not only in the combinations described as an example, but also in other combinations or alone, without going beyond the scope of the present invention.

A description of a device related to the features of the method is valid, regarding these features, similarly for the corresponding method, as long as the features of the method represent corresponding functional features of the described device.

1. A planning device (P) for determining control data for a treatment device (1) for preparing a surgical tattoo of the cornea (22) of an eye (3) of a patient, the planning device (P) being designed to generate the control data for a treatment device (1) that comprises a laser device (L), which cuts the tissue of the cornea by applying a pulsed laser beam (2), the laser beam (2) being focused on target points (6) arranged in a pattern in the cornea (22), said planning device (P) comprising:
an interface (S) for the introduction of measuring data regarding parameters of the eye (3) and functional data regarding the functions to be performed by the tattoo of the cornea (22) of the eye (3),
wherein said interface defines, from the introduced measuring data and functional data, a globally annular surface (32) that is located inside the cornea (22) and that is limited by a globally circular interior edge (15) with an interior diameter and a globally circular exterior edge (16) with an exterior diameter, the interior diameter and the exterior diameter being positioned in an identified position and/or on an identified structure of the surface of the cornea (23) and the annular surface (32) having a distance and an incline relative to the surface of the cornea (23), and
wherein said interface generates, for this globally annular surface (32), a set of control data for the control of the laser device (L), which defines, in the cornea (22), a pattern of target points (6), which are found in the annular surface (32), and which are arranged such that the annular surface (32) is made, after the application of the pulsed laser beam (2) according to the set of control data, in the form of a cutting surface,
wherein the annular surface (32) containing, at each target point (6), a perforation zone (4) in which, during the application of the pulsed laser beam (2), the corneal tissue is cut,
wherein the perforation zones (4) of adjacent target points (6) being able to overlap partially or completely; and
wherein the exterior edge (16) of the globally annular surface (32) having, in the macroscopic sense, a constant distance relative to the exterior edge of the iris (14).

2. (canceled)

3. The planning device (P) according to claim 1, wherein the annular surface (32) after the application of the pulsed laser beam (2) has a different perforation (6) in different zones of the annular surface (32), which is preferably determined as a function of the desired degree of coloration of the tattoo in different zones of the annular surface.

4. The planning device (P) according to claim 1, wherein the annular surface (32) is designed and the pigmented dye is designed such that, after the application of the pulsed laser beam (2) and after the absorption of the pigmented dye (2) in the annular surface (32), the latter has an absorption greater than or equal to 50%, preferably an absorption greater than or equal to 80%.

5. The planning device (P) according to claim 1, wherein the annular surface (32) comprises a spared zone (17) that is preferably arranged at the exterior edge (16) of the annular surface (32); the spared zone (17) preferably having a surface area greater than 0.2 mm².

6-7. (canceled)

8. The planning device (P) according to claim 1, wherein the center of the circular interior edge and the center of the circular exterior edge do not coincide.

9. (canceled)

10. The planning device (P) according to claim 1, wherein said interface defines, from the introduced measurement data and functional data, at least one additional globally annular surface (33), which is located inside the cornea (22), the at least two annular surfaces (32, 33) having different distances relative to the surface of the cornea (23) and the at least two annular surfaces (32, 33) being superimposed and the at least two annular surfaces (32, 33) preferably being centered relative to one another regarding their interior edges (15) or their exterior edges (16), preferably the annular surface (33), which has the smallest distance relative to the surface of the cornea (23), has the smallest gap between the exterior diameter and the interior diameter.

11. The planning device (P) according to claim 1, wherein said interface further defines, from introduced measurement data and functional data, at least one access surface (35), which goes from the surface of the cornea (23) to the annular surface (32), and
wherein said interface generates, for this access surface (35), a set of control data for controlling the laser device (L), which defines, in the cornea (22), a pattern of target points (6), which are found in the access surface (35) and which are arranged such that the access surface (35) is made, after the application of the pulsed laser beam (2) according to the set of control data, in the form of an access cutting surface, the target points (6) of the access surface (35) having a radial distance relative to the center of the interior edge (15) that is greater than half of the interior diameter of the annular surface (32).

12. The planning device (P) according to claim 11, wherein the access surface (35) is oriented radially and its radial extension is smaller than the gap between half of the exterior diameter and half of the interior diameter or the access surface (35) being oriented along the exterior edge (16) or parallel to the exterior edge (16).

13-14. (canceled)

15. The planning device (P) according to claim 1, wherein the annular surface (32) is positioned using a recorded image.

16. The planning device (P) according to claim 1, further comprising:
a measuring device connected to the interface that generates the measurement data from a measurement of the eye (3) and that introduces them into the planning device (P), the measurement device (M) optionally comprising one or several of the following devices:
autorefractometer, refractometer, keratometer, aberrometer, wave front measuring device, optical coherence tomograph (OCT)

17. The planning device (P) according to claim 1, further comprising:
a data link or data medium is provided for transmitting the set of control data from the planning device (P) to the laser device (L).

18. The planning device (P) according to claim 1, further comprising:
a display device is provided for the visual representation of the control data of the set of control data and an input device for the subsequent modification of the set of control data.

19. The planning device (P) according to claim 1, wherein the planning device (P) takes into account, during the generation of the set of control data containing the pattern of the target points (6), a deformation of the cornea (22) of the eye (3), which occurs during the application of the pulsed laser beam (2), more particularly using an interface with the patient (13), optionally a contact lens or a liquid interface with the patient, such that the defined annular surface (32) finds itself in the nondeformed cornea (22).

20. A treatment device (1) for the surgical tattooing of the cornea (22) of an eye of a patient, comprising:
an interface (S) for the introduction of measurement data regarding the parameters of the eye (3) and functional data regarding the functions to be fulfilled by the tattoo of the cornea (22) of the eye (3),
a laser device (L) that cuts the tissue of the cornea by applying a pulsed laser beam (2), the laser beam (2) being focused on target points (6) found in a pattern of the cornea (22), and
a planning device (P) according to claim 1.

21. The treatment device (1) according to claim 20, further comprising:
a laser beam source for producing a pulsed laser beam, more particularly a femtosecond laser source, a lens for the focusing of the pulsed laser beam in the cornea, an x-y scanning system and a z scanning system as well as a control system.

22. A method for preparing and generating control data for a treatment device (1) allowing the surgical tattooing of the cornea (22) of an eye (3) of a patient, which comprises a laser device (L), which cuts the tissue of the cornea by applying a pulsed laser beam (2), the laser beam (L) focusing, during its operation, the laser beam (2) according to the control data on target points (6) found in a pattern in the cornea (22), the method comprising the following steps:
determining measurement data regarding the parameters of the eye (3) and functional data regarding the functions to be fulfilled by the tattooing of the eye (3),
defining a globally annular surface (32) from measurement data and functional data,
wherein the annular surface (32) is located inside the cornea (22) and being limited by a globally circular interior edge (15) with an interior diameter and a globally circular exterior edge (16) with an exterior diameter,
wherein the interior diameter and the exterior diameter is positioned at an identified point and/or in line with an identified structure on the surface of the cornea (23), and
wherein the annular surface (32) has a distance and an incline relative to the surface of the cornea (23),
defining a pattern of target points (6) in the cornea (22),
wherein the target points (6) are located in the globally annular surface (32) and being arranged such that the annular surface (32) is made during the application of the laser beam (2) according to the control data in the form of a cutting surface,
wherein the annular surface (32) contains, at each target point (6), a perforation zone (4) in which, during the application of the pulsed laser beam (2), the tissue of the cornea is cut, the perforation zones (4) of adjacent target points (6) partially or completely crossing one another, and
wherein the exterior edge (16) of the globally annular surface (32) has a constant distance, in the macroscopic sense, relative to the exterior edge (14) of the iris, and
generating a set of control data containing the two- or three-dimensional pattern for the control of the laser device (L).

23. The method according to claim 22, wherein the annular surface (32) is further defined or the pattern of target points (6) in the cornea (22) is defined such that at least one of the following statements is relevant:
wherein the target points in the annular surface (32) are arranged such that the annular surface (32) is further designed, after the application of the pulsed laser beam (2), for the absorption of an appropriate pigmented dye;
wherein the points in the annular surface (32) are arranged such that the annular surface (32) has, after the application of the pulsed laser beam (2), different perforations (4) in different zones of the annular surface (32);
wherein the annular surface (32) comprises a spared zone (17), which is preferably arranged on the exterior edge (16) of the annular surface (32); the spared zone (17) preferably having a surface area greater than 0.2 mm²;
wherein the annular surface (32) comprises an interior edge (15) with an interior diameter greater than 4 mm or the annular surface (32) comprises an interior edge (15) with an interior diameter smaller than 3 mm;
wherein the center of the circular interior edge (15) and the center of the circular exterior edge (16) do not coincide;
wherein the exterior edge (16) and/or the interior edge (15) does or do not have a smooth curve;
wherein the annular surface (32) is designed, after the application of the pulsed laser beam (2), for housing an implant;
wherein the annular surface (32) is defined in a lenticule (28) in the tissue of the cornea of the eye (3), which is explanted after the application of the pulsed laser beam (2); and
wherein the annular surface (32) is positioned using a recorded image.

24. The method according to claim 22, wherein, from the measurement data and functional data, an additional annular surface (33) is defined, which is located inside the cornea (22), the at least two annular surfaces (32, 33) having different distances relative to the surface of the cornea (23), and the at least two annular surfaces (32, 33) overlapping each other and the at least two annular surfaces (32, 33) preferably being centered relative to one another with respect to their interior edges (15) or their exterior edges (16), preferably the annular surface (33), which has the smallest distance relative to the surface of the cornea (23), has the smallest gap between the exterior diameter and the interior diameter.

25. The method according to claim 22, wherein, from the introduced measurement data and functional data, at least one access surface (35) is defined, which goes from the surface of the cornea (23) to the annular surface (32), and, for this access surface (35), a set of control data for controlling the laser device (L) is generated, which defines, in the cornea (22), a pattern of target points (6), which are found in the access surface (35) and which are arranged such that the access surface (35) is made, after the application of the pulsed laser beam (2) according to the set of control data, in the form of an access cutting surface, the target points (6) of the access surface (35) having a radial distance relative to the center of the interior edge (15) that is greater than half of the interior diameter of the annular surface (32);
also optionally characterized in that the access surface (35) is oriented radially and its radial extension is smaller than the gap between half of the exterior diameter and half of the interior diameter or the access surface (35) being oriented along the exterior edge (16) or parallel to the exterior edge (16).

26. The method according to
claim 22, wherein measurement data are generated from a measurement of the eye (3), the used measurement device (M) optionally being one or several of the following devices: autorefractometer, refractometer, keratometer, aberrometer, wave front measuring device, optical coherence tomograph (OCT), and/or
wherein the generated control data are transmitted to the treatment device (1); these optionally being transmitted via a data link or a data medium to the laser device (L).

27. The method according to claim 22, wherein, during the generation of the set of control data containing the pattern of the target points (6), a deformation of the cornea (22) of the eye (3) is taken into account, which occurs during the application of the pulsed laser beam (2), more particularly using an interface with the patient (13), optionally a contact lens or a liquid interface with the patient, such that the defined annular surface (32) is found in the nondeformed cornea (22).

28-29. (canceled)

30. A method for surgical tattooing of the cornea (22) of an eye (3) of a patient, with the following steps:
execution of the method for preparing and generating control data for a treatment device (1) allowing the surgical tattooing of the cornea (22) of an eye (3) of a patient, which comprises a laser device (L), which cuts the tissue of the cornea by applying a pulsed laser beam (2), according to claim 22,
surgical laser treatment of the cornea (22) of the eye (3) with the treatment device (1) using the generated control data,
optional mechanical cutting of the tissue bridges (5) remaining with the laser treatment in the surfaces made during the surgical laser treatment, for example with a surgical tool such as a flap lifter; and
injection of at least one pigmented dye into the annular surface (32), for example, using access surfaces (35).
